(19) United States
(12) Patent Application Publication
(10) Pub. No.: US 2012/0101507 A1
(43) Pub. Date: Apr. 26, 2012

(54) Anvil Placement System

(76) Inventor: Dale Sloan, Fort Wayne, IN (US)

(21) Appl. No.: 12/667,087

(22) PCT Filed: Jun. 30, 2008
(86) PCT No.: PCT/US2008/068772

§ 371 Date: May 21, 2010

Related U.S. Application Data
(60) Provisional application No. 60/947,164, filed on Jun. 29, 2007.

(51) Int. Cl. A61B 17/00

(52) U.S. Cl. 606/108

(57) ABSTRACT

Disclosed are versions of an anvil placement rod for use in bariatric surgical procedures and a split sheath catheter used to facilitate insertion of an anvil and circular stapler into a body cavity.

### PRIORITY

The application claims priority from the disclosure of U.S. Provisional Patent Application Ser. No. 60/947,164, entitled “Anvil Placement System,” filed Jun. 29, 2007, which is herein incorporated by reference in its entirety.

### FIELD OF THE INVENTION

Versions of the present invention relate to minimally invasive medical procedures and, more particularly, to minimally invasive bariatric or weight loss surgical procedures.

### BACKGROUND

Individuals who need weight loss surgery at times select the roux-en-y gastric bypass procedure. During the procedure, a small gastric pouch is created using a stapler technique. This pouch is separated entirely from the rest of the stomach. In order to reconnect the pouch to the small intestine, an opening is made between the pouch and the small intestine. This opening can be made either by suturing the pouch to the small intestine, using a linear stapler and suturing, or using a circular stapler only.

There are various advantages and disadvantages to each technique. Suturing has the disadvantage of being technically more difficult and slower than the stapling technique. It has the advantage of placing the connection between the pouch and small intestine in the most dependent and optimal position.

The circular staple technique has the advantage of being relatively quick and easy to perform. It does however require an anvil to be placed through the abdominal wall or through the mouth and down the esophagus and then into proper position inside the pouch.

### BRIEF DESCRIPTION OF THE FIGURES

In accordance with versions herein, it is believed the present invention will be better understood from the following description taken in conjunction with the accompanying drawings. The drawings and detailed description that follow are intended to be merely illustrative and are not intended to limit the scope of the invention.

FIG. 1 depicts a plan view of one version of an anvil placement rod shown in a hooked configuration.

FIG. 2 depicts a plan view of the anvil placement rod of FIG. 1 shown in a substantially straight or linear configuration for insertion through an access port.

FIG. 3 depicts a plan view of the anvil placement rod of FIG. 1 shown in a hooked configuration after a wire having an attached suture has been introduced into the rod.

FIG. 4 depicts a plan view of the anvil placement rod of FIG. 1 shown in a hooked configuration with the wire having the attached suture passing through the distal end of the rod.

FIG. 5 depicts a plan view of the anvil placement rod, wire, and suture of FIG. 4 shown after placement within a patient's stomach, where the suture is shown grasped by a pair of forceps.

FIG. 6 depicts a plan view of the suture of FIG. 5, where one end of the suture is shown grasped by forceps after removal of the anvil placement rod and wire of FIG. 5 and the other end of the suture is shown associated with an anvil.

FIG. 7 depicts a plan view of the suture and anvil of FIG. 6, where the anvil is shown pulled into position through the patient's stomach.

FIG. 8 depicts a plan view of the anvil of FIG. 7 after removal of the suture.

FIG. 9 depicts a plan view of one version of a split sheath catheter shown in an unrolled configuration.

FIG. 10 depicts a plan view of the split sheath catheter of FIG. 9 shown in a rolled configuration with a flap valve open.

FIG. 11 depicts a plan view of the split sheath catheter of FIG. 9 shown in a rolled configuration with the flap valve closed and welded.

FIG. 12 depicts a side view of the split sheath catheter of FIG. 11 shown inserted through an access port into a body cavity.

FIG. 13 depicts a cross-sectional view taken along line 12-12 of the split sheath catheter shown in FIG. 12.

FIG. 14 depicts a side view of the split sheath catheter of FIG. 11 shown with the access port of FIG. 12 removed from the body cavity.

FIG. 15 depicts a side view of the split sheath catheter of FIG. 11 shown with a dilation rod inserted therein.

FIG. 16 depicts a side view of the split sheath catheter of FIG. 11 shown with an anvil inserted partially therethrough.

FIG. 17 illustrates one version of a circular stapler anvil shown prior to insertion through the split sheath catheter of FIG. 11.

FIG. 18 illustrates a side view of the dilation rod of FIG. 15 shown associated with the anvil of FIG. 17.

FIG. 19 illustrates a side view of the dilation rod of FIG. 18 shown with the anvil inserted into a receptacle of the dilation rod.

FIG. 20 illustrates a side view of the dilation rod of FIG. 19 shown inserted into the split sheath catheter of FIG. 11.

FIG. 21 illustrates a side view of the anvil of FIG. 20 shown inside a body cavity after being released from the dilation rod.

FIG. 22 depicts a side view of one version of a circular stapler shown prior to insertion through the split sheath catheter of FIG. 11.

FIG. 23 depicts a side view of the circular stapler of FIG. 22 shown partially inserted into the split sheath catheter.

FIG. 24 depicts the circular stapler of FIG. 23 shown fully inserted through the split sheath catheter.

FIG. 25 depicts a plan view of an alternate version of an anvil placement rod shown in a hooked configuration.

FIG. 26 depicts a plan view of the anvil placement rod of FIG. 25 shown in a substantially straight or linear configuration for insertion through an access port.

FIG. 27 depicts a plan view of an alternate version of an anvil placement rod shown in a hooked configuration with a guide wire extended.

FIG. 28 depicts a plan view of an alternate version of an anvil placement rod shown in a hooked configuration with a guide wire retracted.

FIG. 29 illustrates a side view of an alternate version of a tapered dilation rod 450.

FIG. 30 depicts an alternate use of the split sheath catheter, following the step shown in FIG. 15, where a rolled mesh material is inserted through the split sheath catheter of FIG. 11.

FIG. 31 depicts a side view of the split sheath catheter of FIG. 30 shown with the dilation rod of FIG. 15 urging the rolled mesh material into the body cavity.

FIG. 32 depicts a side view of the split sheath catheter of FIG. 31 shown with the rolled mesh material of FIG. 30 fully inserted into the body cavity and where the access port of FIG. 12 is shown reintroduced into the split sheath catheter.

FIG. 33 depicts a side view of the split sheath catheter of FIG. 32 shown with the access port of FIG. 12 inserted through the wall of the body cavity and with the split sheath catheter partially withdrawn from around the access port.

FIG. 34 depicts a side view of the access port of FIG. 33 shown with the split sheath catheter of FIG. 11 fully removed from the body cavity.

### DETAILED DESCRIPTION OF THE INVENTION

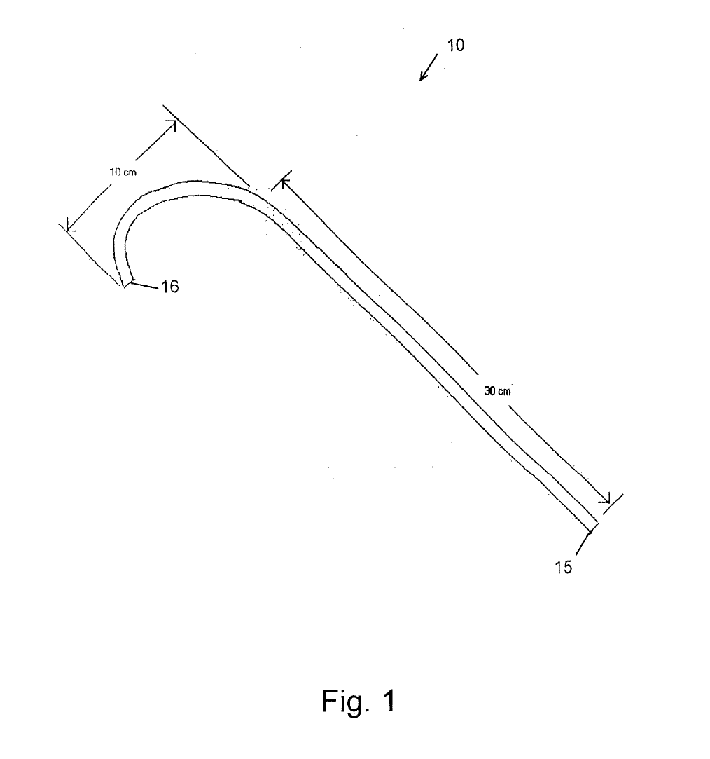

FIG. 1 depicts a plan view of one version of an anvil placement rod 10 shown in a hooked configuration. The anvil placement rod 10 may be a hollow plastic rod having a first end 15 and a second end 16 measuring from 3-7 mm in diameter shaped in a hook fashion in its resting state.

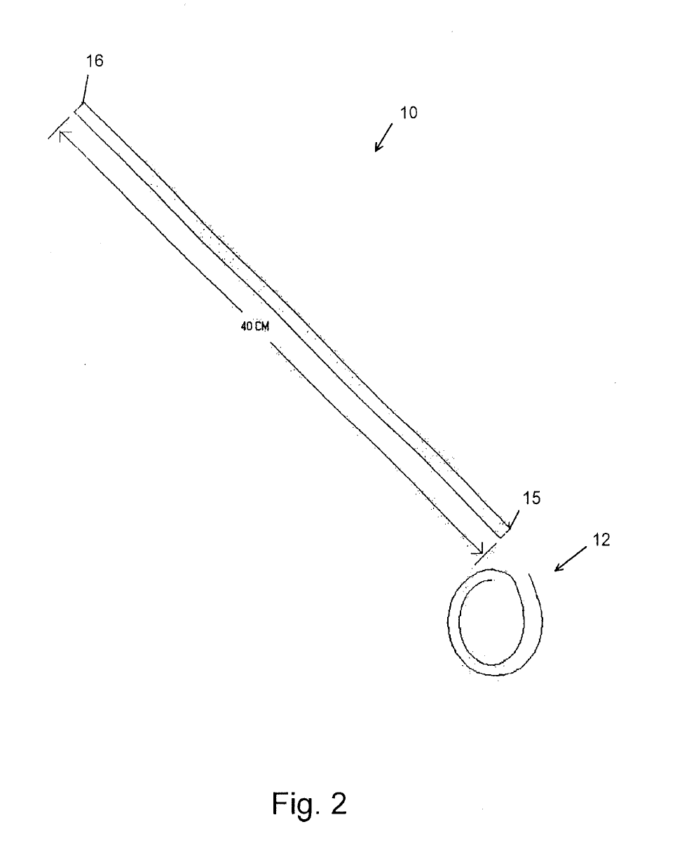

FIG. 2 depicts a plan view of the anvil placement rod 10 of FIG. 1 shown in a substantially straight or linear configuration for insertion through an access port. The rod 10 is shown in a straightened state for insertion through a 12 mm abdominal wall port. A wire 12 is shown prior to insertion through the rod 10.

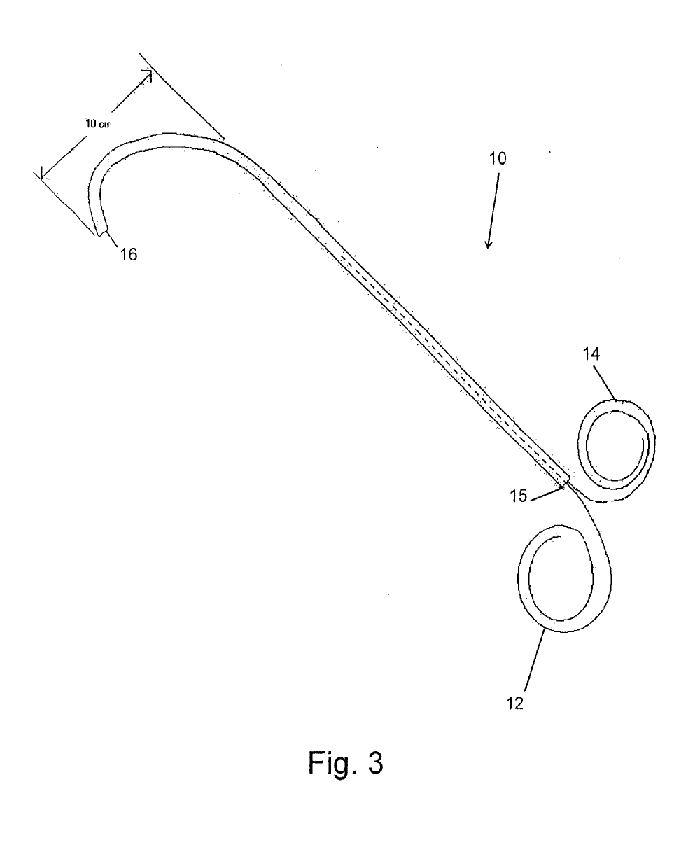

FIG. 3 depicts a plan view of the anvil placement rod 10 of FIG. 1 shown in a hooked configuration after the wire 12 having an attached suture 14 has been introduced into the rod 10. The rod 10 is shown with a wire 12 and suture 14 threaded inside and a coil of each ready to be advanced through the center of the rod 10 to the tip of the distal or second end 16.

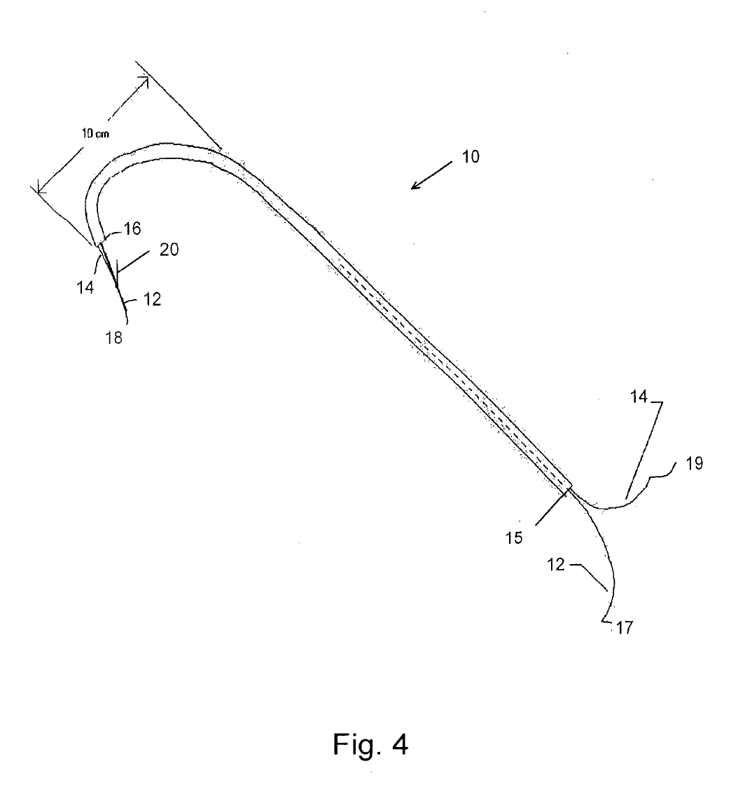

FIG. 4 depicts a plan view of the anvil placement rod 10 of FIG. 1 shown in a hooked configuration with the wire 12 having the attached suture 14 passing through the distal or second end 16 of the rod 10. The wire has a first end 17 and a second end 18 and the suture has a first end 19 and a second end 20. The second end 18 of the suture 14 is attached to the wire 12 at the second end 18.

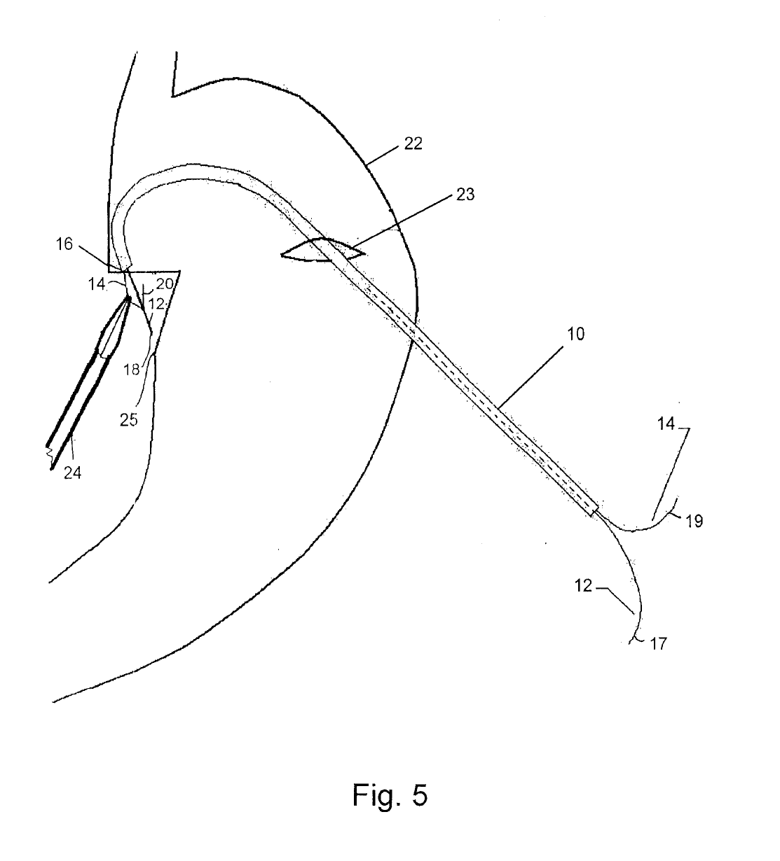

FIG. 5 depicts a plan view of the anvil placement rod 10, wire 12, and suture 14 of FIG. 4 shown after placement within a patient's stomach 22, where the suture 14 is shown grasped by a pair of forceps 24. FIG. 5 shows the rod 10 with the wire 12 and suture 12 pushed beyond the end or second end 16 of the rod 10 so that the suture 14 can be separated from the wire 12. FIG. 5 shows the rod 10 after it has been placed through a port (not shown) in the straightened configuration and then placed through an opening 23 made in the anterior wall of the stomach 22 after the rod 10 resumes a hook configuration. The rod 10 is pushed to the proper position in the end of the new gastric pouch. In one version, the gastric pouch formation has already been started by a horizontal application of an endoscopic linear stapler (not shown) to divide the stomach 22 along the lesser curvature. Once the rod 10 is in place, the wire 12 and suture 14 are advanced beyond the second end 16 of the rod 10, through the horizontal staple line 25 and into view of the surgeon.

In the illustrated version, the surgeon is then able to grasp the suture 14 with a laparoscopic forceps 24 and separate the suture 14 from the wire 12. The rod 10 and wire 12 are then removed from the abdomen leaving the suture 14 inside the stomach 22.

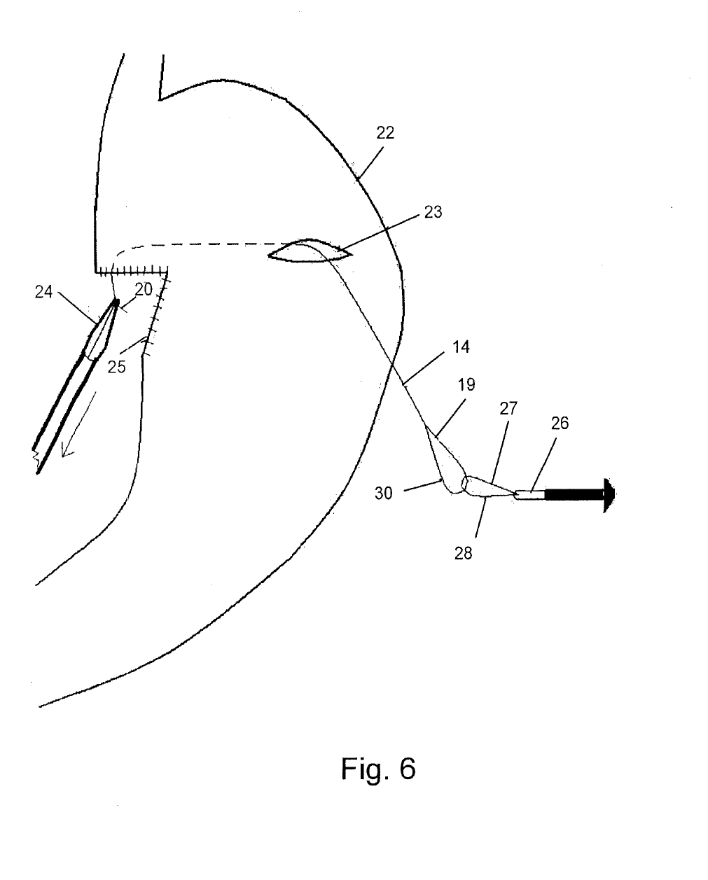

FIG. 6 depicts a plan view of the suture 14 of FIG. 5, where the second end 16 of the suture 14 is shown grasped by forceps 24 after removal of the anvil placement rod 10 and wire 12 of FIG. 5. The first end 19 of the suture 14 is shown associated with an anvil 26. FIG. 6 shows the anvil 26 after it has been placed inside the abdomen (potentially using the SCIPD system to be described later). It is attached to the suture 14 by passing a loop 28 in an anvil suture 27 through the loop 20 in the stomach suture 14. Alternatively, the anvil 26 can be attached to the wire 12 after the rod 10 is removed from the port (not shown) and before the proximal or first end 17 of the wire 12 is pulled inside the abdomen. The anvil 26 can now be pulled through the opening 23 in the stomach 22 and out through the staple line 25 in the end of the gastric pouch.

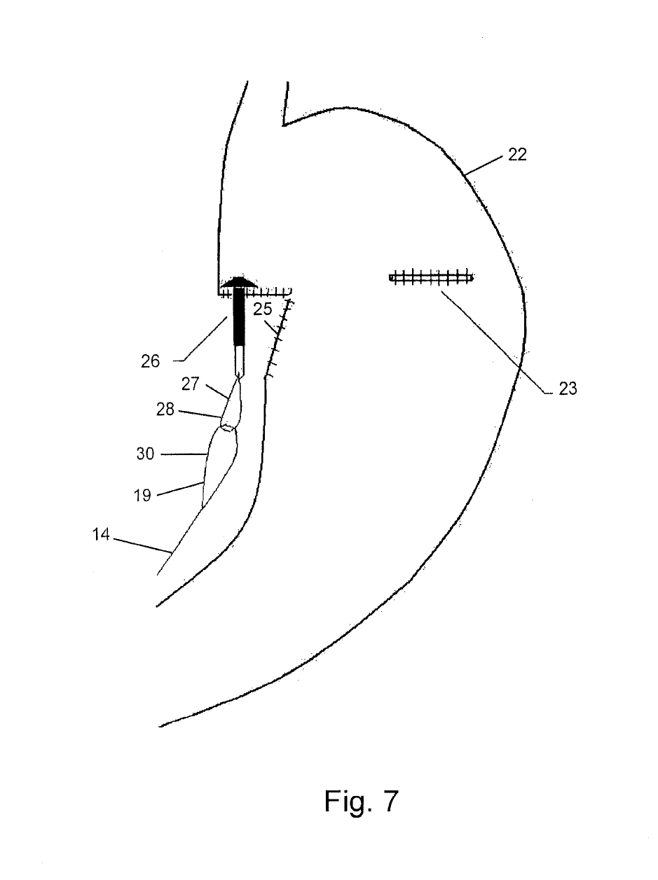

FIG. 7 depicts a plan view of the suture 14 and anvil 26 of FIG. 6, where the anvil 26 is shown pulled into position through the patient's stomach 22. FIG. 7 shows the anvil 26 in proper position in the end of the pouch ready for the pouch creation to be completed.

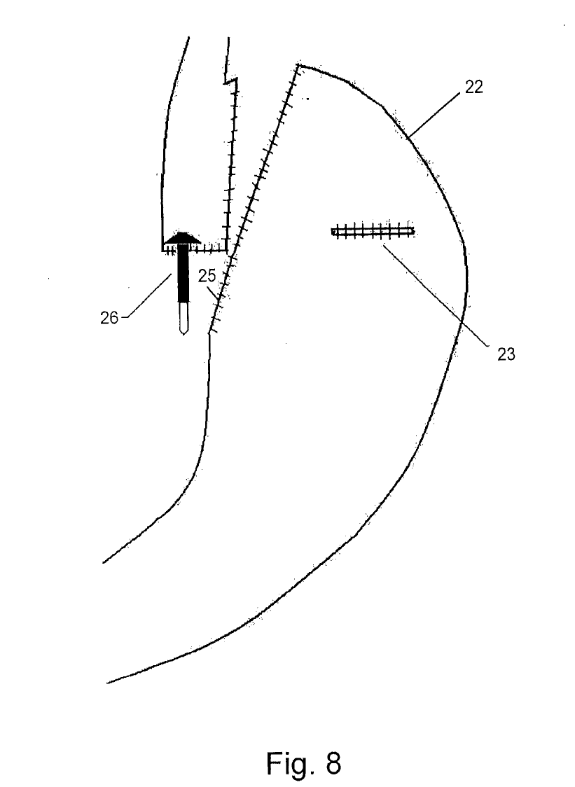

FIG. 8 depicts a plan view of the anvil 26 of FIG. 7 after removal of the suture 14. FIG. 8 shows the anvil 26 in position after the pouch has been created ready for anastamosis.

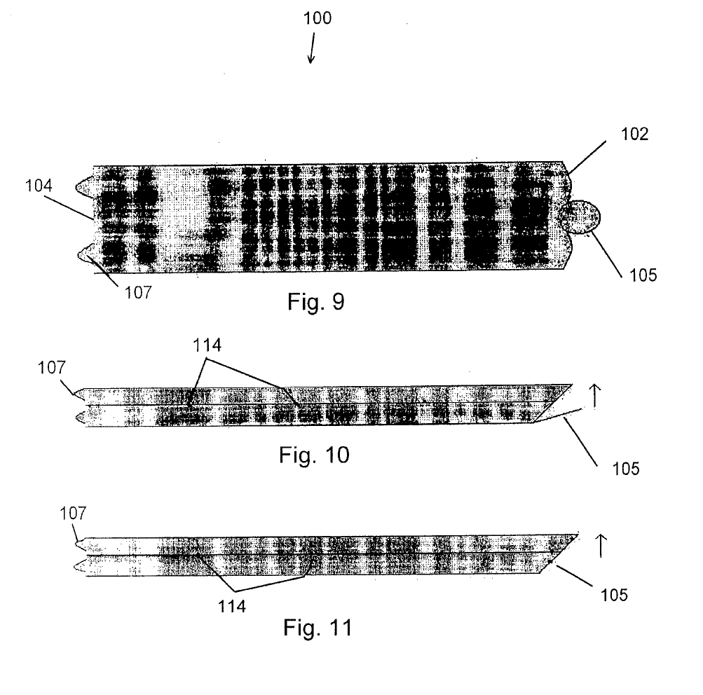

FIG. 9 depicts a plan view of one version of a split sheath catheter 100 shown in an unrolled configuration, where the catheter 100 has a first end 102 and a second end 104.

FIG. 10 depicts a plan view of the split sheath catheter 100 of FIG. 9 shown in a rolled configuration with a flap valve 105 open. The catheter 100 further includes insertion or entry tabs 107 at the second end 104. The catheter 100 is maintained in a rolled configuration with a plurality of tack welds 114. In one version, the catheter 100 is configured from an inelastic material and is configured to expand as overlapping portions of the catheter expand across one another. The catheter 100 may also be configured from an elastic material to expand to accept objects having varying sizes.

The catheter 100 may also be retained in a rolled configuration by extruding the catheter or by heat treating a sheet of material into a rolled configuration such that the material forms a contiguous catheter. Such a catheter may include a tear line comprised of perforations, a seam, a perforated line, tack welds, and/or an area having a reduced wall thickness to facilitate separation or unrolling of the catheter for removal around an access port or other device. The tear line may be solid or perforated. The tear line may be configured such that the catheter 100 retains its integrity while stretching during the insertion of objects having various sizes, where the catheter is then separate into a sheet of material by being torn along the tear line by the clinician. The tear line may be configured such that once a small tear is made in the tear line it is relatively easy to unroll the catheter along the seam or tear line.

The flap valve 105 is configured to retain insufflation gas, such as carbon dioxide, from leaking through the catheter 100 during use. It will be appreciated that the catheter 100 may have any suitable leakage prevention member to reduce gas leakage from the body 118 including an external stopper located at the second end 104 of the catheter, a tapered end at the first end 102 that is occlusive when nothing is being passed through the catheter 100, or the like. A one-way valve may also be provided at the first end 102, second end 104, in the region where the catheter 100 enters the body cavity 118, and/or in any other suitable location. Multiple valves configured as an airlock may allow the catheter 100 to be stretched during insertion of an object without releasing substantial amounts of insufflation gas.

FIG. 11 depicts a plan view of the split sheath catheter 100 of FIG. 9 shown in a rolled configuration with the flap valve 105 closed and welded.

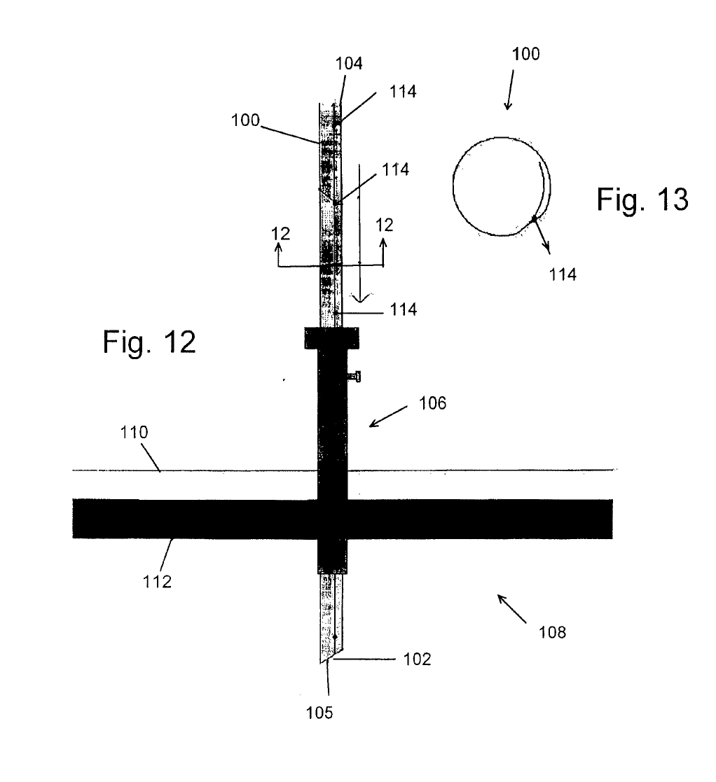

FIG. 12 depicts a side view of the split sheath catheter 100 of FIG. 11 shown inserted through an access port 106 into a body cavity 108. The access port 106 is inserted through a layer of skin 110 and a layer of muscle 112.

FIG. 13 depicts a cross-sectional view taken along line 12-12 of the split sheath catheter 100 shown in FIG. 12 illustrating one of a plurality of tack welds 114.

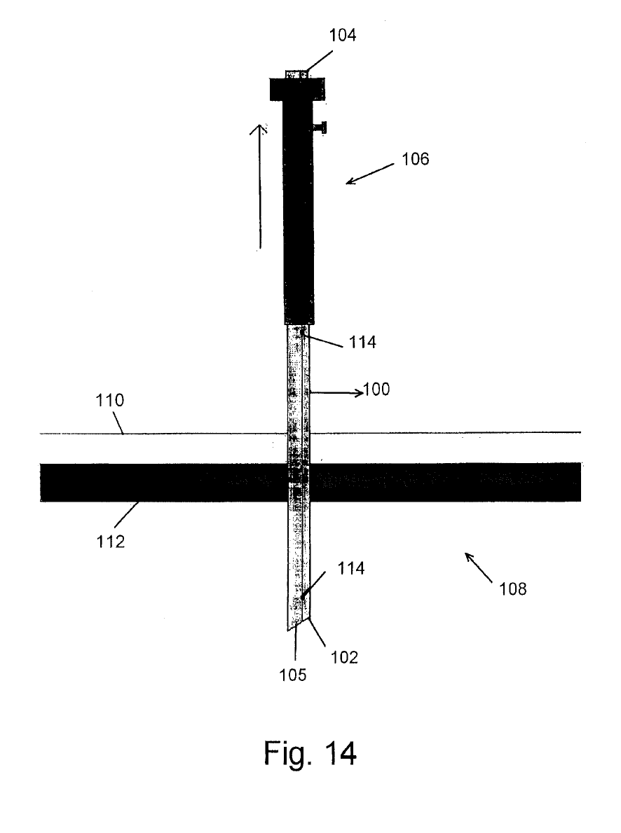

FIG. 14 depicts a side view of the split sheath catheter 100 of FIG. 11 shown with the access port 106 of FIG. 12 removed from the body cavity 108. FIG. 14 shows the port 106 being removed leaving the catheter 100 in place through the skin 110 and muscle 112 layers.

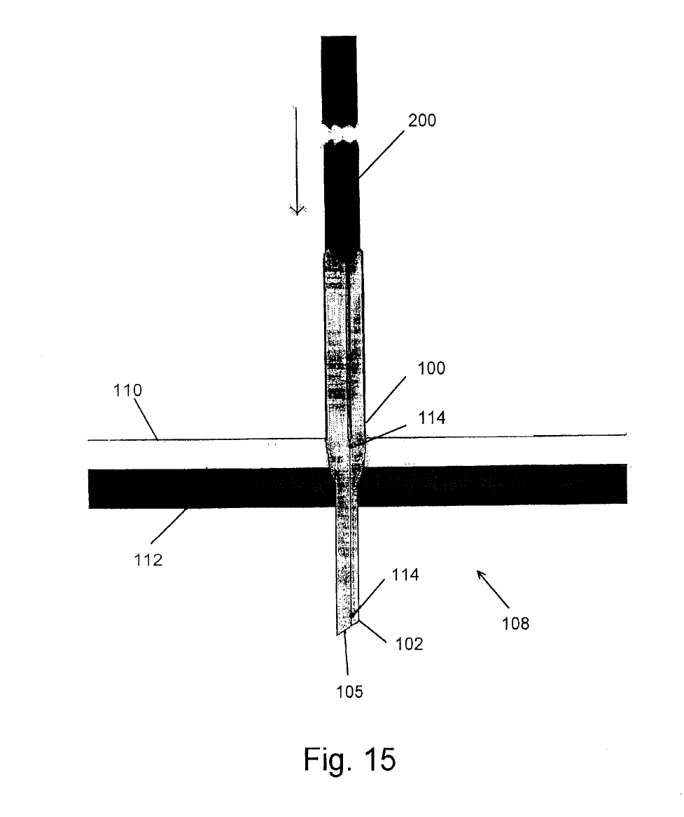

FIG. 15 depicts a side view of the split sheath catheter 100 of FIG. 11 shown with a dilation rod 200 inserted therein.

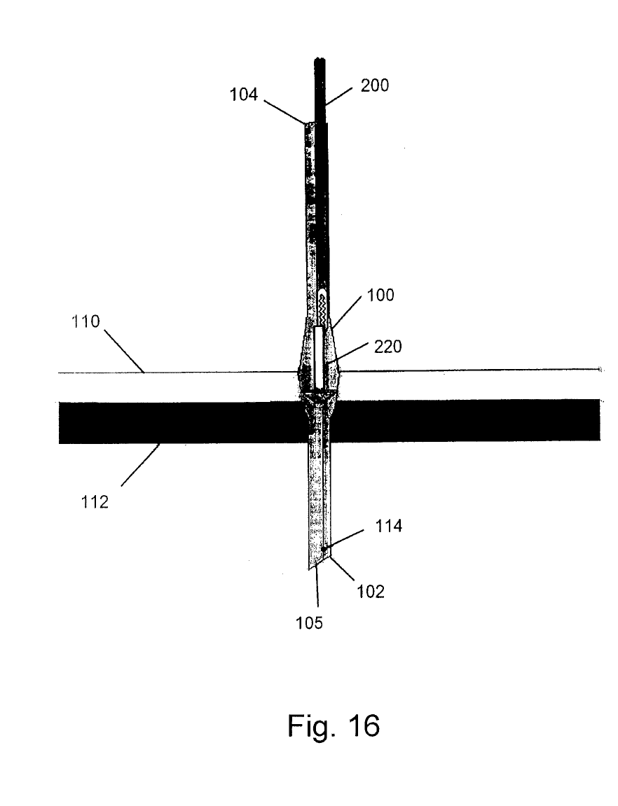

FIG. 16 depicts a side view of the split sheath catheter of FIG. 11 shown with an anvil 220 inserted partially therethrough. FIG. 16 shows one method of anvil 220 placement inside the abdomen using a laparoscopic grasper 222 to push the anvil 220 through the split catheter 100.

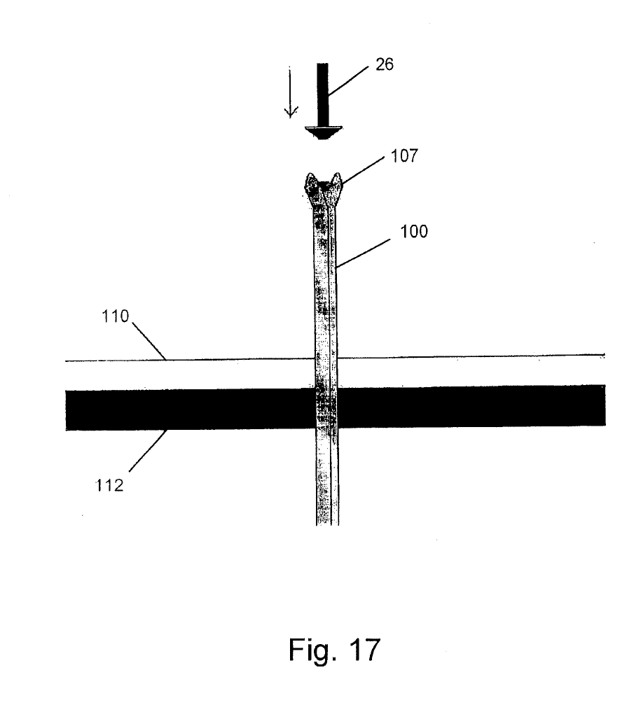

FIG. 17 illustrates one version of a circular stapler anvil 222 shown prior to insertion through the split sheath catheter 100 of FIG. 11.

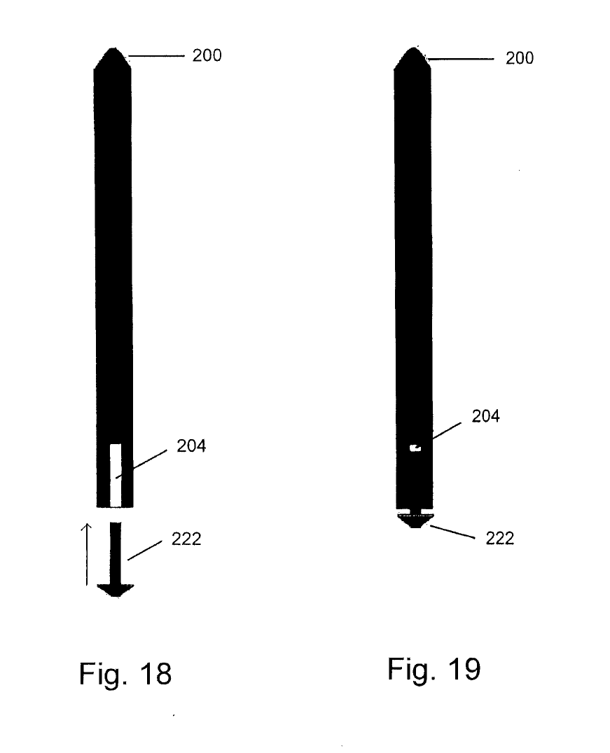

FIG. 18 illustrates a side view of the dilation rod 200 of FIG. 15 shown associated with the anvil 222 of FIG. 17.

FIG. 19 illustrates a side view of the dilation rod 200 of FIG. 18 shown with the anvil 222 inserted into a receptacle 204 of the dilation rod 200.

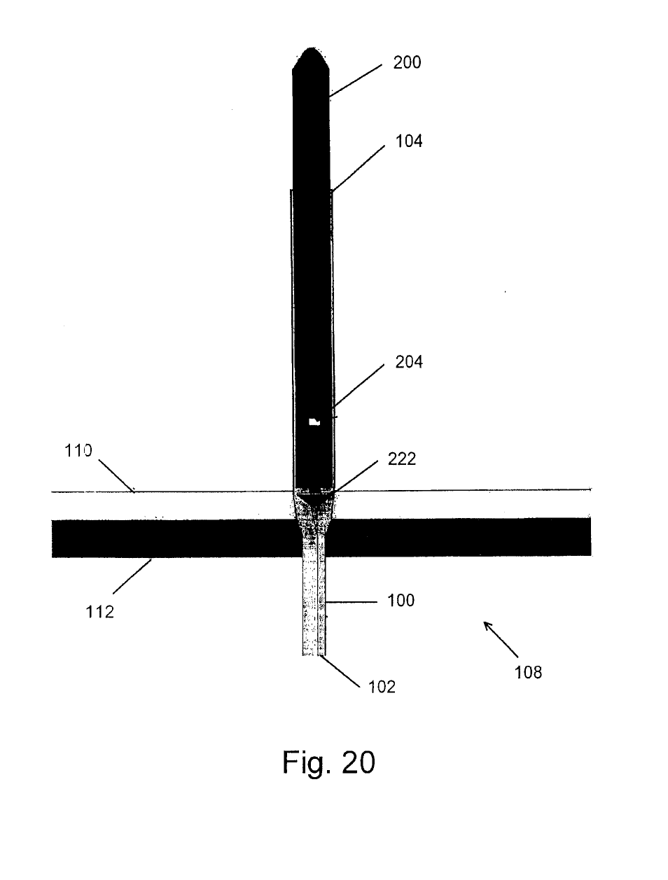

FIG. 20 illustrates a side view of the dilation rod 200 of FIG. 19 shown inserted into the split sheath catheter 100 of FIG. 11.

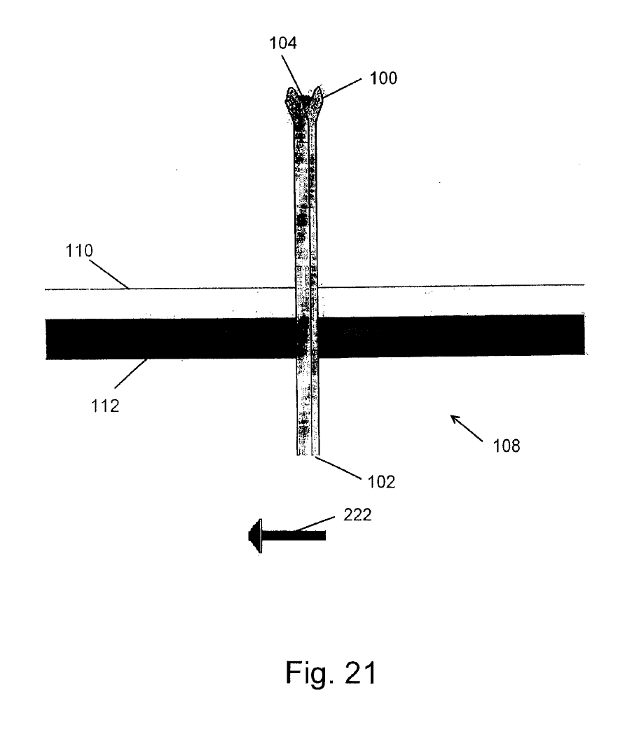

FIG. 21 illustrates a side view of the anvil 222 of FIG. 20 shown inside a body cavity 108 after being released from the dilation rod 200.

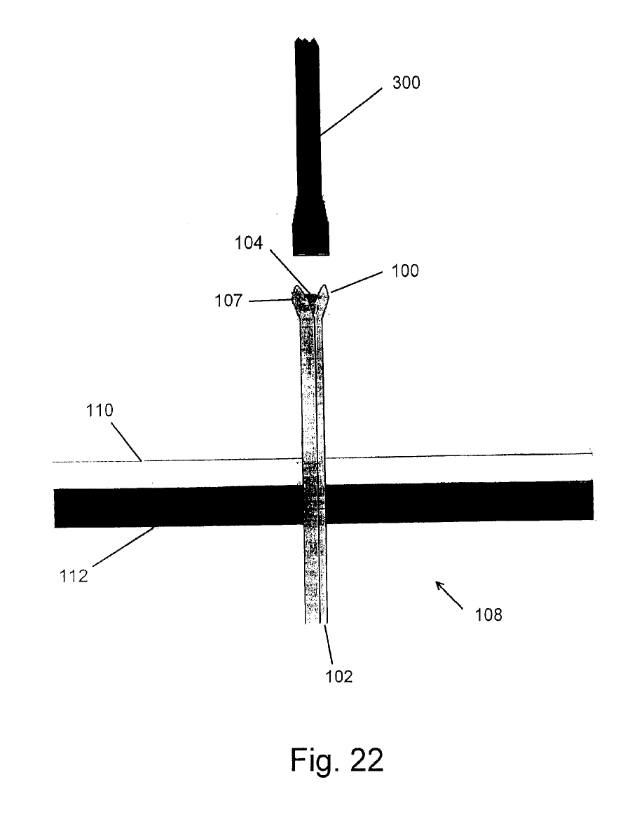

FIG. 22 depicts a side view of one version of a circular stapler 300 shown prior to insertion through the split sheath catheter 100 of FIG. 11.

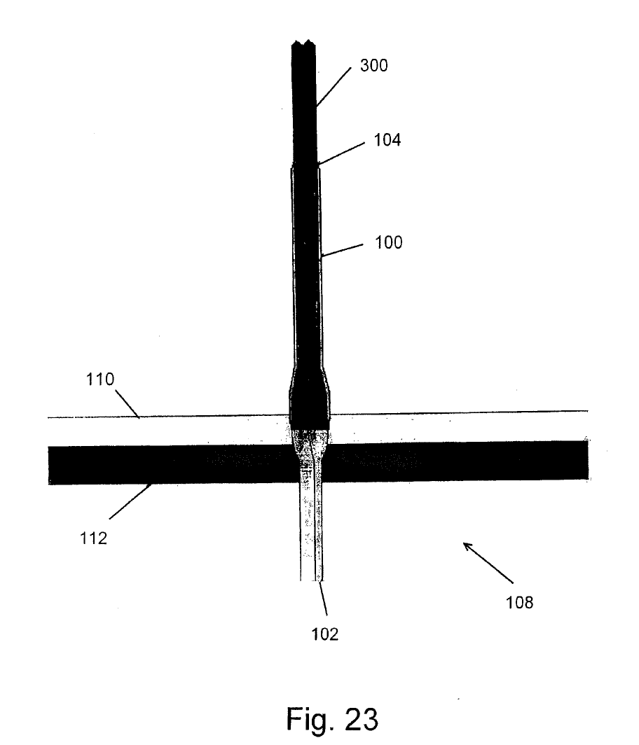

FIG. 23 depicts a side view of the circular stapler 300 of FIG. 22 shown partially inserted into the split sheath catheter 100.

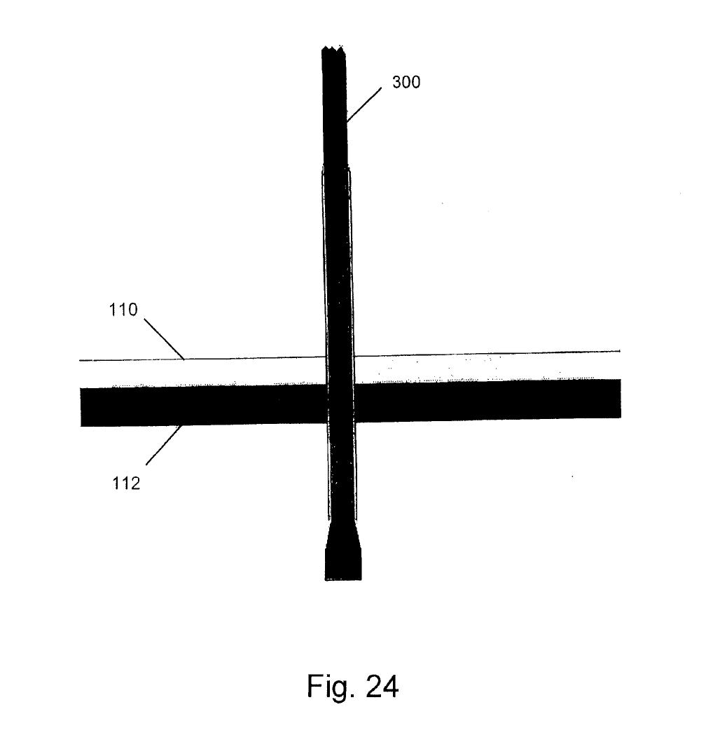

FIG. 24 depicts the circular stapler 300 of FIG. 23 shown fully inserted through the split sheath catheter 100. FIG. 24 shows the circular stapler 300 completely through the catheter 100 with, for example, the end inside the abdomen ready to be placed inside the bowel and then coupled with the anvil 222 shown in FIG. 21.

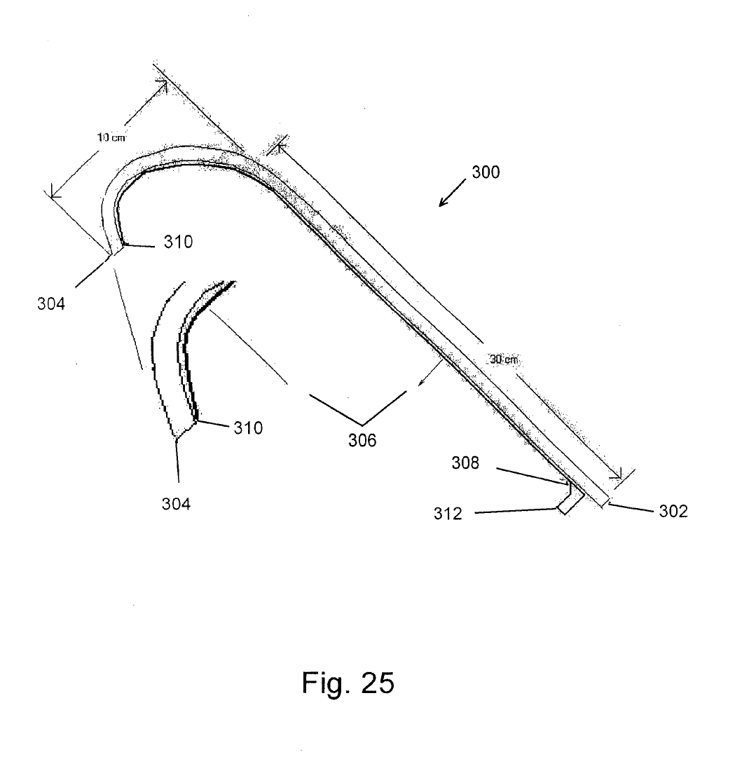

FIG. 25 depicts a plan view of an alternate version of an anvil placement rod 300 shown in a hooked configuration. The rod 300 has a first end 302 and a second end 304. The rod 300 includes a deflection cable 306 having a first end 308 and a second end 310. The first end 308 is coupled with a deflection trigger 312 located at the first end 302 of the rod 300. The second end 310 of the deflection cable is associated with the second end 304 of the rod 300. In the illustrated version the trigger 312 is drawn proximally to deflect the second end 304 of the rod 300.

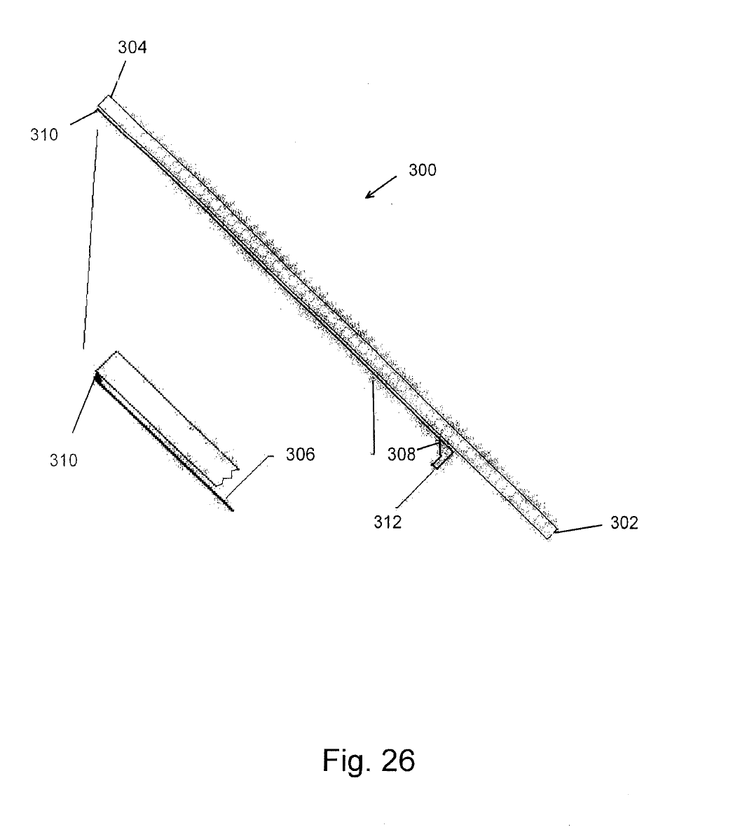

FIG. 26 depicts a plan view of the anvil placement rod 300 of FIG. 25 shown in a substantially straight or linear configuration for insertion through an access port (not shown). The trigger 312 is shown in a distal position where the rod 300 is returned to a linear configuration for insertion through the access port.

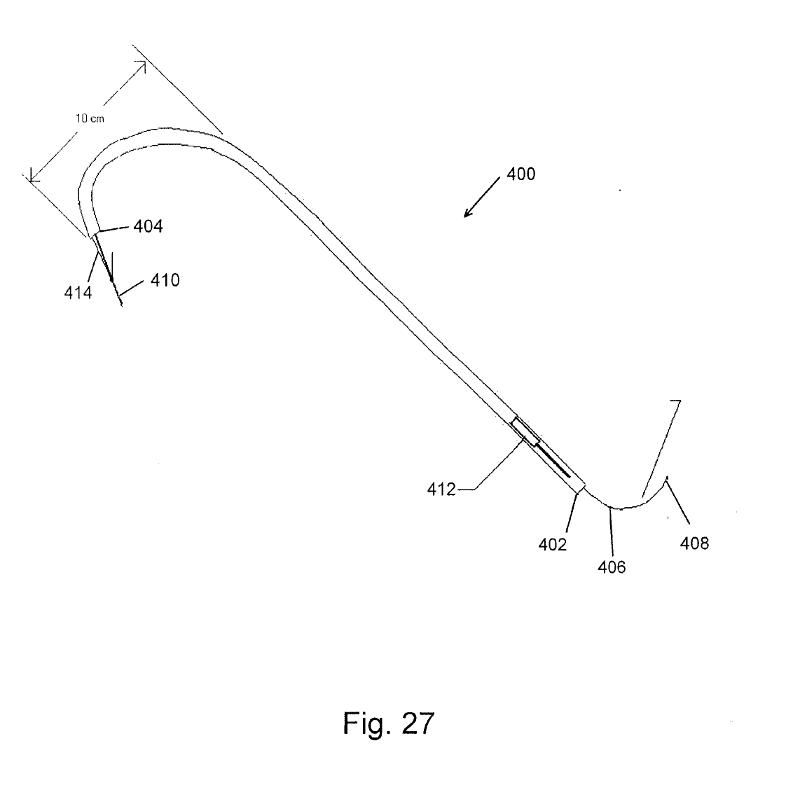

FIG. 27 depicts a plan view of an alternate version of an anvil placement rod 400 shown in a hooked configuration with a guide wire 406 extended. The rod 400 has a first end 402 and a second end 404. The rod 400 includes a wire 406 having a first end 408 and a second end 410. The first end 408 is coupled with a finger slide 412 located at the first end 402 of the rod 400. The wire 406 is associated with a suture 414. In the illustrated version, the slide 412 is drawn proximally to retract the wire 406 and is urged distally to extend the wire 406.

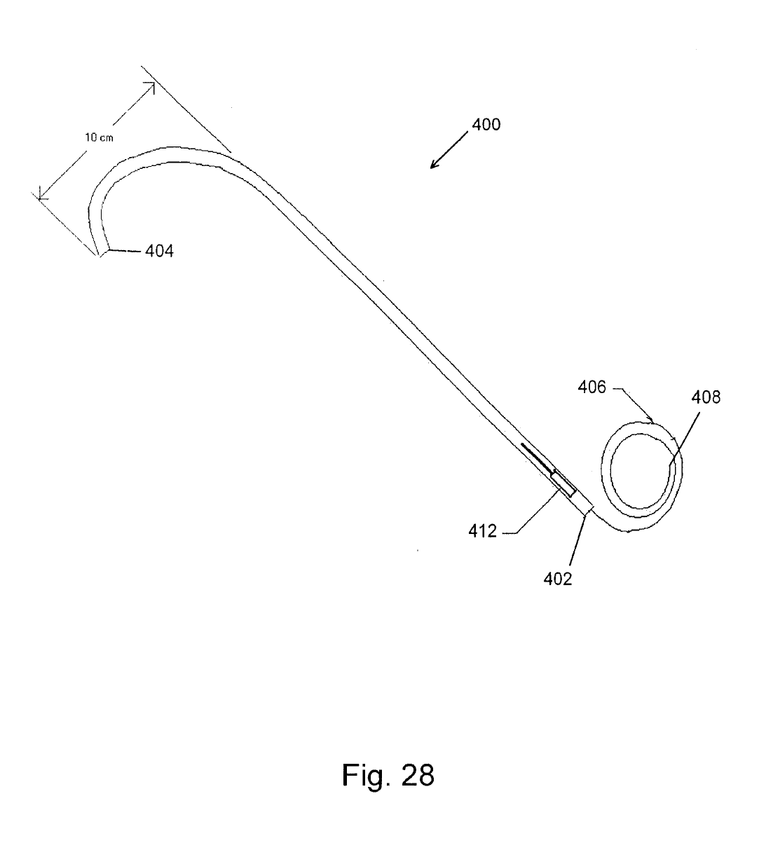

FIG. 28 depicts a plan view the anvil placement rod 400 shown in a hooked configuration with the guide wire 406 retracted.

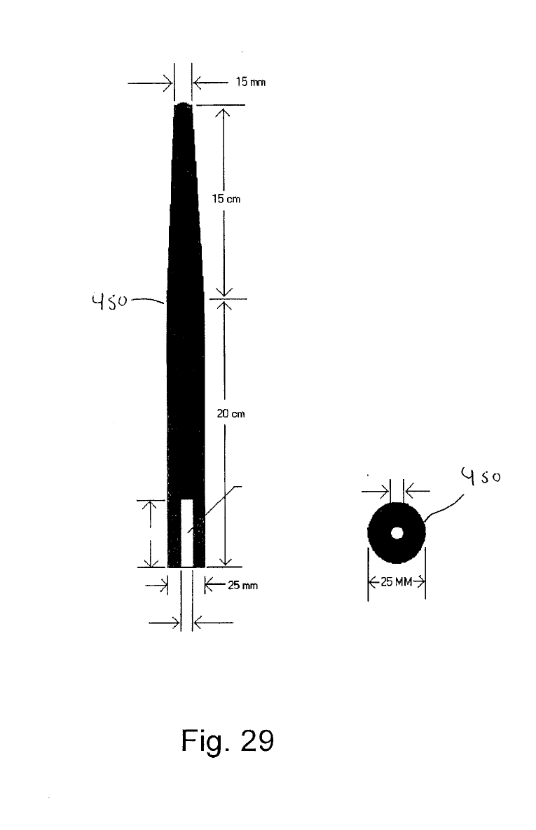

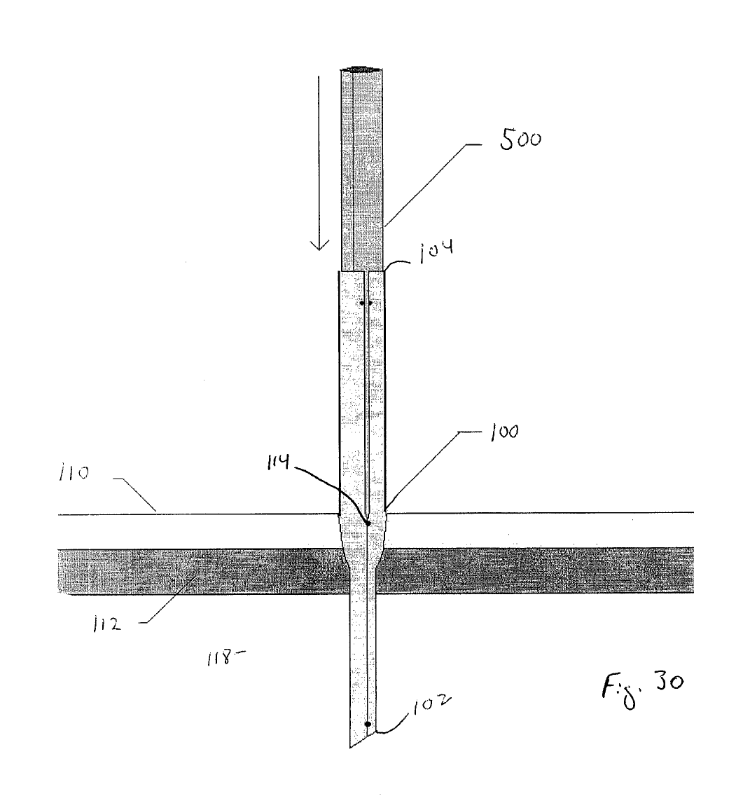

FIG. 29 illustrates a side view of an alternate version of a tapered dilation rod 450. The dilation rod 450 is configured to accept an anvil and is tapered to facilitate insertion. The tapered dilation rod 450 is configured with a tapered end that can be inserted first through the catheter 100 of FIG. 11 to expand the access point into the body cavity 118. After dilation, the dilation rod 450 may be turned and inserted to deliver the anvil into the body cavity 118.

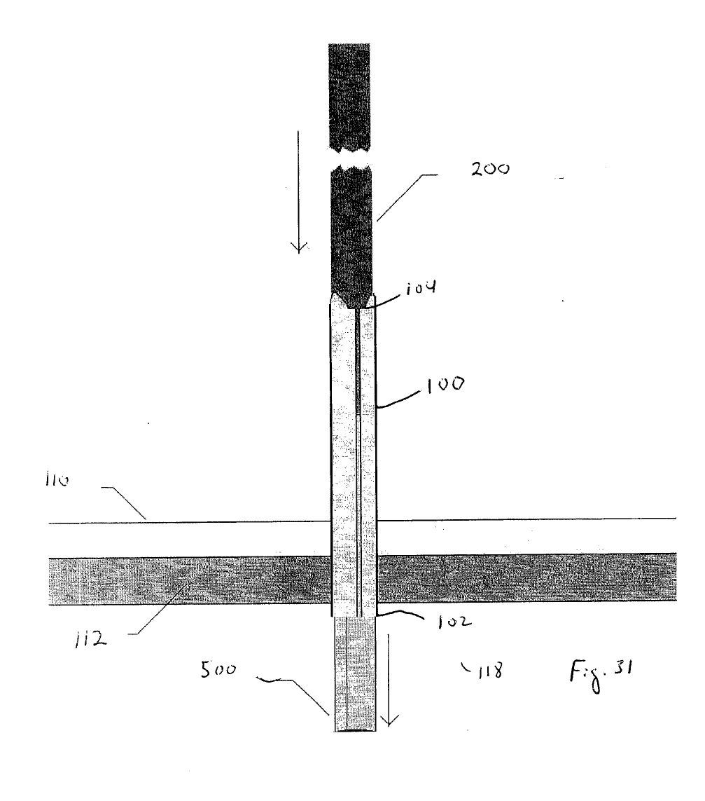

FIG. 30 depicts an alternate use of the split sheath catheter 100, following the step shown in FIG. 15, where a rolled mesh material 500 is inserted through the split sheath catheter 100 of FIG. 11.

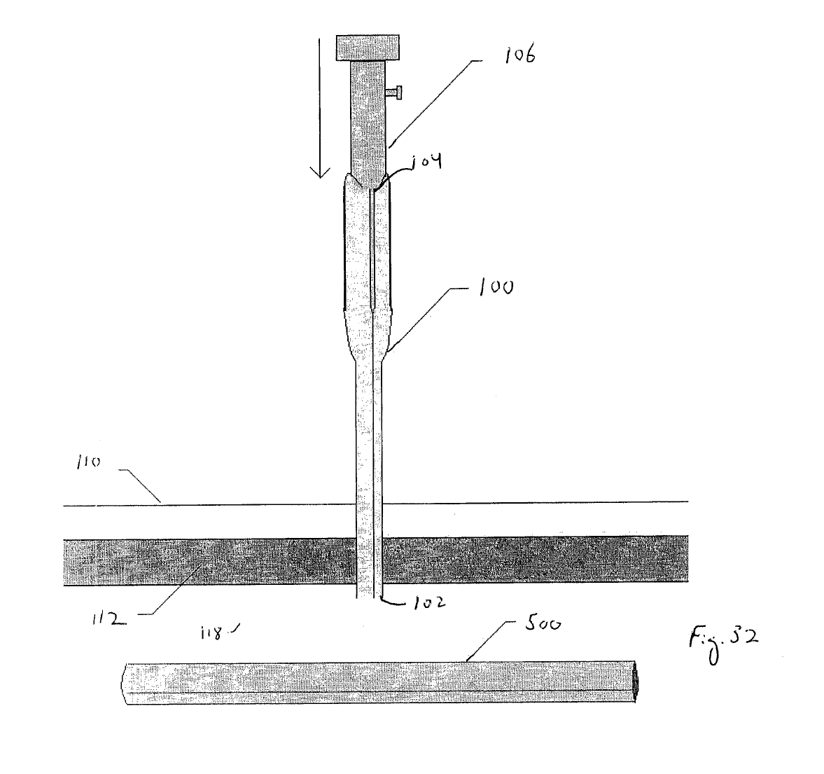

FIG. 31 depicts a side view of the split sheath catheter 100 of FIG. 30 shown with the dilation rod 200 of FIG. 15 urging the rolled mesh material 200 into the body cavity 118.

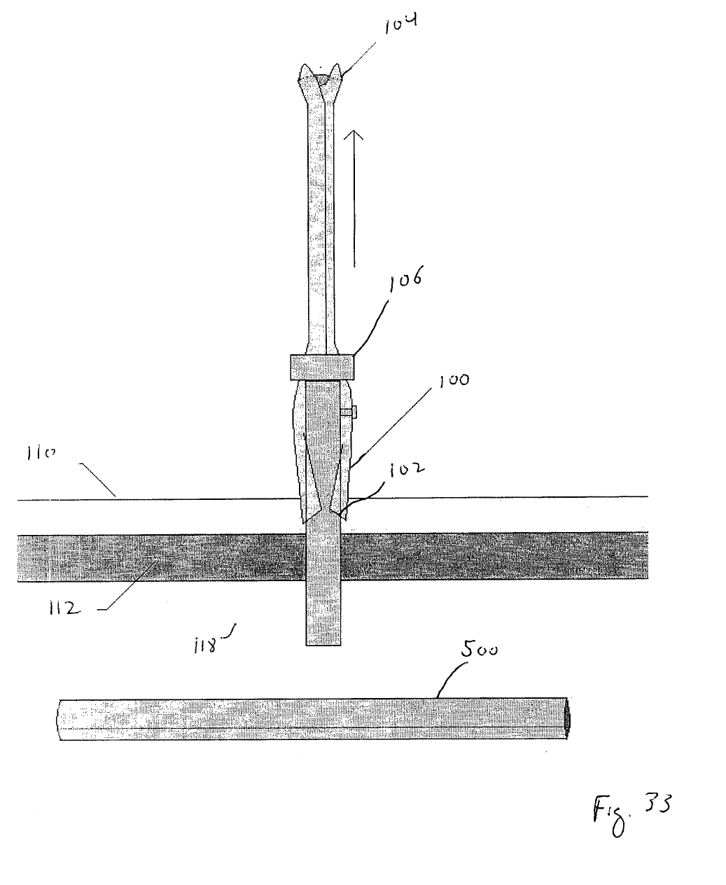

FIG. 32 depicts a side view of the split sheath catheter 100 of FIG. 31 shown with the rolled mesh material 500 of FIG. 30 fully inserted into the body cavity 118 and where the access port 106 of FIG. 12 is shown reintroduced into the split sheath catheter 100.

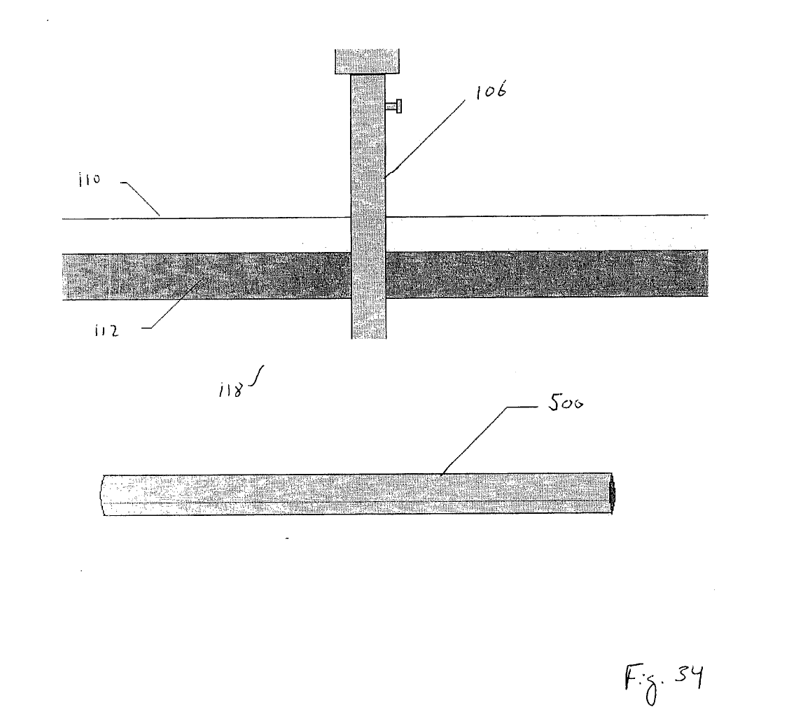

FIG. 33 depicts a side view of the split sheath catheter 100 of FIG. 32 shown with the access port 106 of FIG. 12 inserted through the wall of the body cavity 118 and with the split sheath catheter 100 partially withdrawn from around the access port 106.

FIG. 34 depicts a side view of the access port 106 of FIG. 33 shown with the split sheath catheter 100 of FIG. 11 fully removed from the body cavity 118. In the steps illustrated in FIGS. 30-34 it is shown that any suitable instrument, material, device, implant, pacemaker, gastric band, stapler, anvil, or the like, may be inserted through the split sheath catheter in accordance with versions herein. For example, during a procedure, the access port 106, shown in FIG. 12, may be used as the primary access point for instruments and the like into the abdominal cavity. The abdominal cavity is insufflated with carbon dioxide and the access port 106, which may be a trocar, permits access to the abdominal cavity without releasing gas from within the cavity. The access port 106 may have a diameter that limits the size of instruments and such that can inserted through the access port 106 into the body cavity 118. It may be desirable during the procedure to insert an instrument or device into the body cavity 118 that is larger than the diameter of the access port without releasing a substantial amount of the gas from within the body cavity 118.

FIGS. 30-34 illustrate, in one version how a rolled mesh material, which has a larger diameter than the access port 106, can be inserted into the cavity 118 without causing a substantial amount of carbon dioxide to be released from the cavity. Utilizing a split sheath catheter 100 allows for the sheath to be inserted through the trocar, the trocar to be removed leaving the catheter 100 in place, a large diameter device to be inserted through the catheter 100, the access port 106 to be reintroduced through the catheter 100, and the sheath to be removed from around the access port 106 such that the access port 106 is returned to its initial position. In this manner, a clinician can select a trocar having any suitable diameter, where if a larger diameter device needs to be inserted, the larger diameter device can be inserted with the split sheath catheter 100 without significantly disrupting the procedure. Multiple split sheath catheters may be used during a single procedure as the need arises to insert larger diameter items into the body cavity. Additionally, the split sheath catheter may be used to remove devices, tissue, or the like from inside the body cavity 118 that may be too large to pass through the access port. For example, if during a procedure a cyst is discovered that is larger than the trocar, a split sheath catheter may be used for removal of the cyst in a minimally invasive manner.

Although the rolled mesh material 500 is illustrated by way of example, it will be appreciated that any suitable device, instrument, member, tissue, or the like may be inserted into or removed from the body cavity 118 using the split sheath catheter 100. For example, the split sheath catheter may be used for the introduction of a circular stapler, an anvil, large pieces of mesh for hernia repair, plastic bags for removal of detached organs such as the spleen or an ovary, or gastric bands.

What is claimed is:

1. A medical catheter comprising:
(a) an elongate tube, the tube having a first end and a second end, wherein the elongate tube is configured from a sheet of rolled material having a first edge that forms the first end of the tube, a second edge that forms the second end of the tube, and a third edge extending along the length of the tube that is folded within a fourth edge extending along the length of the tube such that the tube has a substantially cylindrical configuration;
(b) a flap valve positioned at the first end of the elongate tube; and
(c) a plurality of entry tabs positioned at the second end of the elongate tube.

2. The medical catheter of claim 1, wherein the elongate tube comprises at least one tack weld, wherein the at least one tack weld couples the fourth edge of the elongate tube to the sheet to retain the tube in the substantially cylindrical configuration.

3. The medical catheter of claim 2, wherein the substantially cylindrical configuration of the elongate tube has a substantially uniform diameter.

4. The medical catheter of claim 3, wherein the at least one tack weld comprises a plurality of tack welds.

5. The medical catheter of claim 1, wherein the third edge and the fourth edge overlap such that the third edge and the fourth edge are configured to slide relative to one another to accommodate the insertion of relatively large objects.

6. The medical catheter of claim 5, wherein the elongate tube is inelastic.

7. A method comprising the steps of:
providing a split sheath catheter, wherein the split sheath catheter comprises;
(a) an elongate tube, the tube having a first end and a second end, wherein the elongate tube is configured from a sheet of rolled material having a first edge that forms the first end of the tube, a second edge that forms the second end of the tube, and a third edge extending along the length of the tube that is folded within a fourth edge extending along the length of the tube such that the tube has a substantially cylindrical configuration; and
(b) at least one tack weld, wherein the at least one tack weld couples the fourth edge of the elongate tube to the sheet to retain the tube in the substantially cylindrical configuration;
providing a first access port, the first access port having a first end and a second end;
inserting the first end of the first access port into a body cavity of a patient, wherein the second end of the first access port remains outside the body cavity.
inserting the second end of the split sheath catheter through the access port and into the body cavity;
removing the access port from the body cavity, wherein the second end of the split sheath catheter remains within the body cavity and the first end of the split sheath cavity remains outside the body cavity;
inserting a medical device through the split sheath catheter into the body cavity;
removing the medical device from the body cavity through the split sheath catheter;
inserting a second access port through the split sheath catheter such that a first end of the second access port is within the body cavity and a second end of the second access port is outside the body cavity; and
removing the split sheath catheter from the body cavity, wherein the split sheath catheter is removed by breaking the at least one tack weld and removing the split sheath catheter from around the access port such that the access port remains within the body cavity.

8. The method of claim 7, wherein the medical device is an instrument used to delivery an anvil into the body cavity and a circular stapler.

9. The method of claim 7, wherein the split sheath catheter further comprises:
(c) a flap valve positioned at the first end of the elongate tube; and
(d) a plurality of entry tabs positioned at the second end of the elongate tube.

10. The method of claim 7, wherein the body cavity is the abdominal cavity.

11. The method of claim 7, wherein the medical device is an anvil placement rod having a tapered first end and a second end configured to retain a circular stapler anvil.

12. The method of claim 11, wherein the step of inserting the medical device through the split sheath catheter further comprises the steps of
inserting the tapered first end of the anvil placement rod; and
inserting the second end of the anvil placement rod to deliver the circular stapler anvil.

13. The method of claim 7, wherein the first access port and the second access port are the same instrument.

14. The method of claim 7, wherein the split sheath catheter is configured to deliver relatively large materials into the body cavity, where the access port has insufficient diameter to accept the materials.

15. The method of claim 7, wherein the breaking strength of the at least one tack weld is sufficient to resist breaking while instruments are being passing into and out of the body cavity.

16. The method of claim 7, wherein the at least one tack weld comprises a plurality of tack welds.

17. A medical catheter comprising:
(a) an elongate tube, the tube having a first end and a second end, wherein the elongate tube is configured from a sheet of rolled material into a substantially cylindrical configuration;
(b) a tear line, the tear line extending from the first end to the second end of the tube, wherein the tear line is configured such that internal axial pressure separates the tube into the sheet of rolled material; and
(c) a leakage prevention member, wherein the leakage prevention member is configured to prevent insufflation gas from leaking out of an insufflated body cavity.

18. The medical catheter of claim 17, wherein the leakage prevention member is a valve.

19. The medical catheter of claim 17, wherein the tear line is an unperforated seam.

20. The medical catheter of claim 17, wherein the elongate tube is inelastic.
